(19) United States
(12) Patent Application Publication
(10) Pub. No.: US 2022/0175656 A1
(43) Pub. Date: Jun. 9, 2022

(54) Deodorant Composition

(71) Applicant: Jewell Parker, Saint Louis, MO (US)

(72) Inventor: Jewell Parker, Saint Louis, MO (US)

(21) Appl. No.: 17/181,676

(22) Filed: Feb. 22, 2021

Related U.S. Application Data
(60) Provisional application No. 63/123,139, filed on Dec. 9, 2020.

(51) Int. Cl. A61K 8/9789; A61K 8/92; A61K 8/98; A61K 8/60; A61Q 15/00; A61K 8/02; A61K 8/19

(52) U.S. Cl. CPC A61K 8/9789; A61K 8/92; A61K 8/988; A61K 8/60; A61Q 15/00; A61K 2800/43; A61K 8/19; A61K 2800/524; A61K 2800/522; A61K 2800/874; A61K 8/0229

(57) ABSTRACT

A deodorizing composition configured to match the user's skin tone. The deodorizing composition is pigmented to match different skin colors ranging from tan to dark brown to avoid leaving a white residue visible on darker skin or clothing. The deodorizing composition is formulated from a combination of solid, dry, and liquid oils. The solid ingredients comprise a combination of fats and waxes. The dry ingredients are absorbing powders for absorbing sweat and blocking odors. The liquid oils include essential oils for masking odor and carrier oils. The deodorizing composition further comprises a colorant ingredient. The colorant ingredient is addable to the composition in amounts based on a desired darkness of skin pigmentation for the user.

### CROSS-REFERENCE TO RELATED APPLICATION

The present application claims priority to, and the benefit of, U.S. Provisional Application No. 63/123,139, which was filed on Dec. 9, 2020 and is incorporated herein by reference in its entirety.

### FIELD OF THE INVENTION

The present invention generally relates to a deodorant composition, and more specifically to a formulation for a natural deodorant with varying shades of pigmentation. Accordingly, the present specification makes specific reference thereto. However, it is to be appreciated that aspects of the present invention are also equally amenable to other like applications, devices and methods of manufacture.

### BACKGROUND OF THE INVENTION

A deodorant is a chemical substance that may be applied to the human body to prevent or mask body odor that naturally occurs due to the bacterial breakdown of perspiration in the armpits, groin, and feet. Antiperspirants are a subclass of deodorants that block sweat glands to prevent sweating. Antiperspirants are used on a wider range of body parts where sweat would be inconvenient or undesirable. Other types of deodorants allow for sweat glands to produce sweat but prevent the bacterial action on sweat. This works because human sweat only has a noticeable smell when it is decomposed by bacteria.

The human body produces perspiration or sweat from two different types of sweat glands. Eccrine sweat glands which cover much of human skin produce a watery odorless sweat, and apocrine sweat glands in the armpits and groin produce a more oily sweat containing a proportion of waste proteins, fatty acids and carbohydrates that can be metabolized by bacteria to produce compounds that cause body odor.

All types of human perspiration are largely odorless initially until the sweat's organic components are fermented by bacteria that thrive in hot, humid environments common in several body locations. The human underarm is among the most consistently warm areas on the surface of the human body. The sweat glands located there readily provide moisture containing a fraction of organic matter, which when excreted, has a vital cooling effect. When adult armpits are washed with an alkaline soap, the skin loses its protective acid mantle (pH 4.5-6), raising the skin pH and disrupting the skin barrier. Many bacteria are adapted to the slightly alkaline environment within or on the human body and therefore thrive within this elevated pH environment. This makes the skin more susceptible to bacterial colonization. Bacteria on the skin feed on the waste proteins and fatty acids in the sweat from the apocrine glands and on dead skin and hair cells These bacteria then release trans-3-methyl-2-hexenoic acid in their waste, which is the primary cause of body odor.

Most existing deodorants that claim to be clear or invisible often still leave a white residue after use. For men and women of color, this residue can be particularly embarrassing as it stains the skin and clothing. Forgoing the use of deodorant to avoid this residue leads to an increase in odor from sweating. Using existing current residue producing deodorants leads to visible unsightly staining.

Therefore, there exists a long felt need in the art for a deodorant for use by people of darker skin pigmentations. There is also a long felt need for a natural solid deodorant that does not leave an embarrassing white residue under and around the user's armpit. Finally, there is a long felt need in the art for a pigmented natural deodorant that matches skin tones ranging from light tan to dark brown.

In this manner, the improved deodorant composition of the present invention accomplishes all of the forgoing objectives, thereby providing an easy solution maintaining proper hygiene. A primary feature of the present invention is a deodorizing composition configured to match the user's skin tone. Finally, the improved deodorant composition of the present invention is capable of masking body odor safely for people of color who do not want unsightly white residue on their skin.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one embodiment thereof, comprises a deodorizing composition. The deodorizing composition is a natural deodorant pigmented to match different skin colors. The deodorizing composition is formulated for use as a deodorant for individuals with darker skin that wish to avoid a white residue at the sight of application. The residue of the deodorizing composition is formulated to match a plurality of skin colors ranging from light tan to dark brown.

The deodorizing composition comprises at least one base ingredient, at least one dry ingredient, at least one essential oil ingredient, and a colorant. The at least one base ingredient may be a base ingredient formulation. The base ingredient formulation is solid at room temperature and normal atmospheric pressure. The base ingredient formulation comprises at least one fat. The base ingredient formulation may further comprise at least one wax. The base ingredient formulation may comprise yellow African shea butter, white African shea butter, coconut oil, and carnauba wax.

The at least one dry ingredient may be a dry ingredient formulation. The dry ingredient formulation is oil soluble. The dry ingredient formulation may comprise kaolin clay powder, arrow root powder, and magnesium hydroxide powder. The at least one essential oil ingredient may be an essential oil formulation. The essential oil formulation may comprise tea tree oil, lavender oil, honeysuckle oil, grapefruit oil, tangerine oil, lemon Eucalyptus oil, rosemary oil, clary sage oil, or combinations thereof. The colorant is cocoa or caramelized sugar.

The deodorizing composition may further comprise at least one carrier oil. The at least carrier oil may be tamanu oil, sunflower oil, or both. The deodorizing composition may further comprise an odorant. The odorant may be a fragrance. The deodorizing composition may further comprise an antioxidant ingredient. The antioxidant ingredient may be honey. The deodorizing composition may further comprise an antibacterial antifungal ingredient. The antibacterial antifungal ingredient may be honey.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They do not intend as an exhaustive description of the invention or do not limit the scope of the invention. Additionally, an illustrated embodiment need not have all the aspects or advantages shown. Thus, in other embodiments, any of the features described herein from different embodiments may be combined.

The present invention describes a pigmented natural deodorant available in a plurality of different color shades. Solid ingredients, such as natural wax, African shea butters, and coconut oil are melted, combined, and slightly cooled. Next, dry ingredients, such as arrowroot powder kaolin clay, magnesium hydroxide, and cocoa powder or caramelized sugar are added to the melted solids. Then, several essential oils are added. Additional ingredients, such as vitamin E, sunflower oil, and fragrance may also be added. The amount of each ingredient may be dependent on the desired shade of the final composition. The deodorant can be applied as a rolled or wiped solid.

The present invention, in one exemplary embodiment, is a deodorizing composition. The deodorizing composition is a natural deodorant pigmented to match different skin colors. The deodorant composition is formulated for use as a solid roll-on deodorant for use by individuals with darker or more pigmented skin that wish to avoid a white residue at the sight of application to the body. The residue of the deodorizing composition is formulated to match a plurality of skin colors ranging from a lighter tan skin shade to a darker brown. Deodorants require a means of absorbing moisture, controlling odor, and covering or masking residual odor to be effective.

The deodorizing composition comprises at least one base ingredient, at least one dry ingredient, at least one essential oil ingredient, and a colorant. The at least one base ingredient may be a base ingredient formulation. The base ingredient formulation is solid at room temperature and normal atmospheric pressure. The base ingredient formulation comprises at least one fat. The base ingredient formulation may further comprise at least one wax. The base ingredient formulation may comprise yellow African shea butter, white African shea butter, coconut oil, and carnauba wax.

The at least one dry ingredient may be a dry ingredient formulation. The dry ingredient formulation is oil soluble. The dry ingredient formulation may comprise kaolin clay powder, arrow root powder, and magnesium hydroxide powder. The at least one essential oil ingredient may be an essential oil formulation. The essential oil formulation may comprise tea tree oil, lavender oil, honeysuckle oil, grapefruit oil, tangerine oil, lemon Eucalyptus oil, rosemary oil, clary sage oil, or combinations thereof. The colorant is cocoa or caramelized sugar.

The deodorizing composition may further comprise at least one carrier oil. The at least carrier oil may be tamanu oil, sunflower oil, or both. The deodorizing composition may further comprise an odorant. The odorant may be a fragrance. The deodorizing composition may further comprise an antioxidant ingredient. The antioxidant ingredient may be honey. The deodorizing composition may further comprise an antibacterial antifungal ingredient. The antibacterial antifungal ingredient may be honey.

1. Deodorant Ingredients

The following ingredients are being submitted to further define various species of the present disclosure. These examples are intended to be illustrative only and are not intended to limit the scope of the present disclosure. Also, parts and percentages are by equivalent volume units, unless otherwise indicated. Tables 1-4 provide the ingredients by type.

TABLE 1

| Ingredients | Type |
|---|---|
| Yellow African Shea Butter | Solid |
| White African Shea Butter | Solid |
| Coconut Oil | Solid |
| Carnauba Wax | Solid |

The solid ingredients are the base ingredients and may be combined into a base formulation comprised of fats and waxes. The base formulation forms the base of the deodorant formulation in its final solid form. Shea butters are fats that function as carrier oils along with the coconut oil. These ingredients essentially “carry” the other ingredients to the skin and keep them in place.

Carnauba wax is a plant based wax, derived from the leaves of Copernicia prunifera, a plant that grows in northeast Brazil. It is a brittle wax, with a melting point of approximately 82-86° C. which is higher than alternative soft waxes. Carnauba wax is desirable as it creates a thinner final product with little staying power. It also acts as a thickener for the final composition, provides texture, and is a vegan ingredient.

TABLE 2

| Ingredients | Type |
|---|---|
| Cocoa | Dry |
| Caramelized Sugar | Dry |
| Kaolin Clay | Dry |
| Arrowroot | Dry |
| Magnesium Hydroxide | Dry |

The dry ingredients kaolin clay and arrowroot are powders that are employed to absorb wetness and excess moisture. They also are antibacterial and can kill the odor causing bacteria. Magnesium Hydroxide, Mg(OH)₂, is a safe and naturally occurring compound used, at a pharmaceutical grade, in natural deodorant products as an alternative to aluminum, baking soda and other ingredients that are potentially irritating, ineffective, or hazardous. Magnesium Hydroxide does not absorb into the skin. Instead, its main purpose in a natural deodorant is to counteract any odor-causing bacteria on the surface of the skin without impacting the dermis—the layer of skin where sweat glands, pores and hair follicles are contained.

The cocoa or caramelized sugar is used in powder form. The cocoa or caramelized sugar powder is used in varying percentages to create the desired pigmentations of the final composition. More cocoa or caramelized sugar power is used to create a darker pigmentation and less is used to create a lighter pigmentation. Cocoa and caramelized sugar are natural ingredients that is safe for skin contact.

TABLE 3

| Ingredients | Type |
|---|---|
| Tea Tree | Essential Oil |
| Lavender | Essential Oil |
| Honeysuckle | Essential Oil |
| Grapefruit | Essential Oil |
| Tangerine | Essential Oil |
| Lemon Eucalyptus | Essential Oil |
| Rosemary | Essential Oil |
| Clary Sage | Essential Oil |

The essential oils are useful for their antibacterial properties and ability to mask odors. They also can add a natural scent to the deodorant composition. Essential oils have the effect of opening up skin pores unlike antiperspirants. This permits the release of toxins and allows the body to cool off as the individual sweats. They also provide for a variety of customizable scents as they can be used all together, or in different combinations and different amounts depending on the desired scent.

TABLE 4

| Ingredients | Type |
|---|---|
| Tamanu Oil | Carrier Oil |
| Sunflower Oil | Carrier Oil |
| Honey | Antimicrobial |
| Vitamin E | Antioxidant |
| Fragrance | Odorant |

Table 4 illustrates the additional additives for the deodorant composition. Tamanu oil is beneficial as it is easily absorbed into the skin and is thought to reduce odor by preventing bacteria from producing due to its antioxidant and anti-inflammatory properties. Tamanu is a nut oil and not an essential oil. Sunflower oil is another carrier oil for engaging the essential oils. Honey is beneficial for its antibacterial and antifungal properties and may also function as a preservative. Vitamin E has antioxidant properties, acts as a moisturizer, and may increase the shelf life of the deodorant composition. Oxidation is a chemical reaction that creates free radicals, which in turn create a chain reaction which damages oils. The natural oxidation process that occurs in the oils is inhibited by the vitamin E which terminates the oxidation reaction. Fragrance is an additional additive that is used to mask odors and customize a desired final scent for the deodorant composition.

2. Deodorant Formulations

The following examples are being submitted to further define various species of the present disclosure. These examples are intended to be illustrative only and are not intended to limit the scope of the present disclosure. Also, parts and percentages are by volume, unless otherwise indicated. Table 5 provides the components and volume percent for the deodorant composition examples with variation in pigmentation color.

The deodorant composition of the present invention comprises combinations of the base ingredients, the dry absorbent ingredients, carrier oil ingredients, the essential oil ingredients, an antioxidant ingredient, an antibacterial antifungal ingredient, and a colorant ingredient. The parts and percentages are by volume. For exemplary purposes, measurements including tablespoons (tbsp), teaspoons (tsp), dashes, drops, or fractions thereof have been converted to drops wherein one milliliter (mL) is equivalent to approximately 20 drops. Similarly, one tbsp is equivalent to 15 mL or approximately 300 drops, one tsp is equivalent to 5 mL or approximately 20 drops, and one dash is equivalent to approximately 10 drops. Exemplary examples of the deodorant composition are described in Table 5 below.

TABLE 5

| | Formulation | Formulation | Formulation |
|---|---|---|---|
| Chemicals | #1 | #2 | #3 |
| Yellow African Shea Butter | 600 | 600 | 600 |
| White African Shea Butter | 450 | 450 | 450 |
| Organic Virgin Coconut Oil | 100 | 100 | 100 |
| Flaked Carnauba Wax | 600 | 600 | 600 |
| Cocoa or Caramelized Sugar | 25 | 100 | 25-100 |
| Kaolin Clay Powder | 100 | 100 | 100 |
| Arrowroot Powder | 600 | 600 | 600 |
| Magnesium Hydroxide Powder | 100 | 100 | 100 |
| Tea Tree Oil | 6 | 6 | 0-6 |
| Lavender Oil | 6 | 6 | 0-6 |
| Honeysuckle Oil | 4 | 4 | 0-4 |
| Grapefruit Oil | 4 | 4 | 0-4 |
| Tangerine Oil | 4 | 4 | 0-4 |
| Lemon Eucalyptus Oil | 4 | 4 | 0-4 |
| Rosemary Oil | 2 | 2 | 0-2 |
| Clary Sage Oil | 2 | 2 | 0-2 |
| Tamanu Oil | 10 | 10 | 10-20 |
| Sunflower Oil | 25 | 25 | 25 |
| Honey | 10 | 10 | 10-20 |
| Vitamin E | 25 | 25 | 25 |
| Fragrance | 25 | 25 | 0-25 |

Formulation 1 illustrates the lightest pigmented or tan colored version of the deodorant composition. Formulation 2 illustrates the darkest pigmented or brown colored version of the deodorant composition. The darkness of the pigmentation color of the deodorant composition is directly dependent of the percentage of the cocoa or caramelized sugar powder ingredient. Typically, the cocoa or caramelized sugar powder is present in an amount ranging between approximately 0.9 to approximately 3.7% by volume of the total deodorant composition depending on the desired darkness. Formulation 3 illustrates a range for the cocoa or caramelized sugar powder, the essential oils, the tamanu oil, the fragrance, and the honey to account for variations in the overall smell of the deodorant composition.

In another exemplary formulation, the deodorant composition approximately comprises: 2 Tbsp of Yellow African Shea Butter; 1½ Tbsp of White African Shea Butter; 1 Tsp of Organic Virgin Coconut Oil; 2 Tbsp of Flaked Carnauba Wax; ¼-1 Tsp of Cocoa (depending on shade); 1 Tsp of Kaolin Clay Powder; 2 Tbsp of Arrowroot Powder; 1 Tsp of Magnesium Hydroxide Powder; ¼ Tsp of Fragrance; 1 Dash of Honey; 1 Dash of Tamanu Oil; ¼ of Tsp Sunflower Oil; ¼ Tsp of Vitamin E; 6 Drops of Tea Tree Oil; 6 Drops of Lavender Oil; 4 Drops of Honeysuckle; 4 Drops of Grapefruit; 4 Drops of Tangerine; 4 Drops of Lemon Eucalyptus; 2 Drops of Rosemary; and 2 Drops of Clary Sage.

To manufacture the deodorant composition, all of the solid ingredients from Table 1 are melted and then slightly cooled. Next, the dry ingredients from Table 2 are gradually added to the melted solids and uniformly combined. The amount of cocoa or caramelized sugar powder added is dependent on the desired final pigmentation shade with a higher percentage making the deodorant composition darker. Once combined, the other ingredients from Tables 3 and 4 are added in desired amounts depending on the desired formulation and uniformly combined. Finally, the deodorant composition is allowed to cool and is molded for use with an applicator.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A deodorant composition comprising:
at least one base ingredient;
at least one dry absorbent ingredient;
at least one essential oil ingredient; and
a colorant.

2. The deodorant composition of claim 1, wherein the deodorant composition is a roll-on solid.

3. The deodorant composition of claim 1, wherein the colorant tints the deodorant composition to match a plurality of darker skin tones.

4. The deodorant composition of claim 1, wherein the colorant is present in an amount between 0.9 to 3.7% by volume of the total deodorant composition.

5. The deodorant composition of claim 1, wherein the colorant is cocoa or caramelized sugar.

6. The deodorant composition of claim 1, wherein the at least one base ingredient comprises at least one fat.

7. The deodorant composition of claim 1, wherein the at least one base ingredient comprises at least one wax.

8. The deodorant composition of claim 1, wherein the at least one base ingredient is melted and partially cooled prior to combining with the other ingredients.

9. The deodorant composition of claim 1, wherein the at least one dry absorbent ingredient comprises magnesium hydroxide powder.

10. The deodorant composition of claim 1 further comprising at least one carrier oil ingredient.

11. The deodorant composition of claim 10, wherein the at least one carrier oil is sunflower oil.

12. The deodorant composition of claim 1 further comprising at least one preservative ingredient.

13. The deodorant composition of claim 1 further comprising at least one moisturizing ingredient.

14. A deodorant composition comprising:
a base ingredient formulation;
a dry ingredient formulation;
at least one carrier oil;
an essential oil ingredient formulation;
an antioxidant ingredient; and
a colorant ingredient.

15. The deodorant composition of claim 14, wherein the base ingredient composition comprises at least one fat, at least one wax, and at least one oil.

16. The deodorant composition of claim 14, wherein the dry ingredient formulation comprises a plurality of absorbent powders.

17. The deodorant composition of claim 14, wherein the colorant ingredient is cocoa powder.

18. The deodorant composition of claim 14 further comprising an antibacterial antifungal ingredient.

19. The deodorant composition of claim 11, wherein the antibacterial antifungal ingredient is honey.

20. A deodorant composition comprising:
a base ingredient formulation;
a dry ingredient formulation;
at least one carrier oil;
an essential oil ingredient formulation;
an antioxidant ingredient;
an antibacterial antifungal ingredient; and
a colorant powder present in an amount between 0.9 to 3.7% by volume of the total deodorant composition; and
wherein once applied, a residue of the deodorant composition is formulated to match a plurality of darker skin tones in color.
